(19) United States
(12) Patent Application Publication
(10) Pub. No.: US 2024/0350014 A1
(43) Pub. Date: Oct. 24, 2024

(54) SYSTEMS AND METHODS FOR PERFORMING PERIPHERAL VASCULAR IMAGING

(71) Applicants: Anuradha Godavarty, Miami, FL (US); Joshua Daniel Hutcheson, Miami, FL (US); Daniela Leizaola, Miami, FL (US); Kevin Leiva, Miami, FL (US); Valentina Dargam, Miami, FL (US)

(72) Inventors: Anuradha Godavarty, Miami, FL (US); Joshua Daniel Hutcheson, Miami, FL (US); Daniela Leizaola, Miami, FL (US); Kevin Leiva, Miami, FL (US); Valentina Dargam, Miami, FL (US)

(73) Assignee: The Florida International University Board of Trustees, Miami, FL (US)

(21) Appl. No.: 18/306,012

(22) Filed: Apr. 24, 2023

(51) Int. Cl. A61B 5/00

(52) U.S. Cl. CPC A61B 5/0075

(57) ABSTRACT

Systems, devices, and methods for performing peripheral vascular imaging are provided. A continuous wave, non-contact, near-infrared optical scanner (NIROS) can utilize at least one near-infrared (NIR) light. The diffuse reflected NIR signal(s) can be obtained from the surface of the tissue of the mammal being imaged. The signal(s) can be optically filtered and can be detected by an NIR-sensitive image sensor. A graphical user interface (GUI) can be used to automate the acquisition of the spatio-temporal diffuse reflected maps from the NIROS device.

### BACKGROUND

Peripheral vascular imaging is performed to assess the microcirculation and saturation of peripheral tissue. Several non-invasive techniques can measure tissue perfusion in the lower extremities, including hyperspectral imaging (HSI), laser Doppler perfusion monitoring (LDPM), laser speckle contrast imaging (LSCI), near-infrared spectroscopy (NIRS), spectrophotometry, transcutaneous oxygenation measurements (TCOM), and vascular optical tomography imaging. TCOM is a gold-standard approach that is used to quantify skin oxygenation; it assesses how the vasculature responds to an oxygenation demand by inducing tissue heating (or vasodilation) to measure for partial pressure of oxygen (TcPO₂) under the skin. However, the technique is time-consuming (>20 min for a single measurement) and obtains these oxygenation measurements only at discrete locations on the tissue. Additionally, TCOM has >10% intra-operator variability.

Perfusion-based measurements of microcirculatory blood flow are obtained using LDPM and LSCI. While these perfusion-based measurements can be obtained in real-time, they are not capable of imaging a larger peripheral region spatially. Modalities such as HSI, MSI, and NIRS measure tissue oxygenation under the skin. While HSI and MSI provide spatial maps of tissue oxygenation, NIRS techniques provide real-time measurements of tissue oxygenation at discrete point locations. NIRS has previously been used extensively in the diagnosis of peripheral arterial disease (PAD), measuring oxygen saturation in the muscles at single point location via exercise stimulating protocols in the presence of PAD. However, these low-cost NIRS devices and/or approaches are limited to muscle perfusion at point locations, making the approach subjective (as it depends on accurate NIRS probe placement) and inconsistent across patients.

### BRIEF SUMMARY

In view of the issues discussed in the Background, there is a need for imaging modalities that are capable of both spatial and temporal-based peripheral vascular imaging to map microcirculatory physiological changes under the skin across a wide-area of the peripheries and dynamically. Embodiments of the subject invention provide novel and advantageous systems, devices, and methods for performing peripheral vascular imaging (e.g., in a mammal, such as a human or a mouse). A continuous wave, non-contact, near-infrared optical scanner (NIROS) can utilize at least one near-infrared (NIR) light (e.g., light-emitting diode (LED)) (such as multi-wavelength (e.g., dual-wavelength, tri-wavelength, or more) NIR lights (e.g., LEDs)). The diffuse reflected NIR signal(s) can be obtained from the surface of the tissue of the mammal being imaged. The signal(s) can be optically filtered (e.g., through a long pass filter and/or a band-pass filter) and can be detected by an NIR-sensitive image sensor (e.g., a camera, such as a complementary metal oxide semiconductor (CMOS) camera). A graphical user interface (GUI) can be used to automate the acquisition of the (multi-wavelength) spatio-temporal diffuse reflected maps from the NIROS device.

In an embodiment, a system for performing non-contact, peripheral vascular imaging on a subject can comprise: an NIR optical imager (which can be referred to as a NIROS); a (non-transitory) machine-readable medium (e.g., a (non-transitory) computer-readable medium) in operable communication with the NIR optical imager and having a GUI stored thereon; and a processor in operable communication with the machine-readable medium. The NIR optical imager can comprise: at least one NIR light; a long pass (and/or band-pass) filter configured to optically filter ambient light (e.g., to allow only NIR signals through the filter) from tissue of the subject and output filtered NIR signals; and an NIR-sensitive image sensor (e.g., a camera, such as a CMOS camera) configured to detect the filtered NIR signals. The GUI can be configured to automatically acquire spatio-temporal diffuse reflected maps from the NIR optical imager based on the filtered NIR signals detected by the NIR-sensitive image sensor. The at least one NIR light can be a multi-wavelength (e.g., dual-wavelength, tri-wavelength, or more) light. The multi-wavelength light can be configured to emit light at a first wavelength and a second wavelength different from the first wavelength, each of the first wavelength and the second wavelength being in a range of from 650 nanometers (nm) to 950 nm (e.g., 690 nm and 830 nm, respectively). The NIR optical imager can be configured such that the first wavelength and the second wavelength (and/or third wavelength, fourth wavelength, etc. (if present)) are multiplexed at a first temporal frequency and a second temporal frequency (and a third temporal frequency, a fourth temporal frequency, etc.), respectively. The first temporal frequency can be the same as the second temporal frequency. Each of the first temporal frequency and the second temporal frequency can be in a range of, for example, from 0.1 Hertz (Hz) to 100 Hz (e.g., 0.5 Hz to 100 Hz, 0.5 Hz to 10 Hz, or 1 Hz to 2 Hz (such as 1 Hz)). The at least one NIR light can be an LED, a laser diode, or any other suitable NIR light source. The NIR optical imager can further comprise an LED driver configured to multiplex light from the at least one NIR light. The spatio-temporal diffuse reflected maps from two or more wavelengths can be used to obtain an oxy-hemoglobin (HbO) map, a deoxy-hemoglobin (HbR) map, a total hemoglobin (HbT) map, and/or an oxygen saturation (StO₂) map for a region of interest (ROI) of the tissue of the subject. The subject can be, for example, a mammalian subject (e.g., a human subject). The system can further comprise a display in operable communication with the machine-readable medium and upon which the GUI (and therefore the spatio-temporal diffuse reflected maps) is displayed. The NIR optical imager can be a standalone device, a smartphone device, or integrated smartphone device.

In another embodiment, a method for performing non-contact, peripheral vascular imaging on a subject can comprise: providing an NIR optical imager comprising at least one NIR light, a filter (e.g., a long-pass filter or a band-pass filter) configured to optically filter ambient light and allow only NIR light to pass, and an NIR-sensitive image sensor configured to detect NIR signals reflected from tissue of the subject; utilizing the NIR optical imager to scan tissue of the subject in a non-contact manner; acquiring spatio-temporal diffuse reflected maps based on the reflected NIR signals detected by the NIR-sensitive image sensor; generating dynamic maps based on the spatio-temporal diffuse reflected maps; and displaying, via a GUI stored on a machine-readable medium in operable communication with the NIR optical imager, the dynamic maps. The dynamic maps can comprise at least one of an HbO map, an HbR map, an HbT map, and an StO₂ map for an ROI of the tissue of the subject. The subject can be, for example, a mammal subject (e.g., a human subject). The at least one NIR light of the NIR optical imager can be a multi-wavelength light. For example, the multi-wavelength light can be a dual-wavelength light configured to emit light at a first wavelength and a second wavelength different from the first wavelength, and each of the first wavelength and the second wavelength can be in a range of from 650 nm to 950 nm (e.g., 690 nm and 830 nm, respectively). The at least one NIR light of the NIR optical imager can be, for example an LED, a laser diode, or any other suitable NIR light source. The NIR optical imager can further comprise an LED driver configured to multiplex light from the at least one NIR light (with the at least one light being an LED), and the method can further comprise multiplexing the first wavelength and the second wavelength at a first temporal frequency and a second temporal frequency, respectively. The first temporal frequency can be the same as, or different from the second temporal frequency. Each of the first temporal frequency and the second temporal frequency can be in a range of, for example, from 0.1 Hz to 100 Hz (e.g., 0.5 Hz to 100 Hz, 0.5 Hz to 10 Hz, or 1 Hz to 2 Hz (such as 1 Hz)). The multi-wavelength light can be configured to emit light at a first wavelength, a second wavelength different from the first wavelength, and a third wavelength different from the first wavelength and the second wavelength (and a fourth wavelength, etc.). Each of the first wavelength, the second wavelength, and the third wavelength can be in a range of from 650 nanometers (nm) to 950 nm. The method can further comprise multiplexing the first wavelength, the second wavelength, and the third wavelength at a first temporal frequency, a second temporal frequency, and a third temporal frequency, respectively. The first temporal frequency, the second temporal frequency, and the third temporal frequency can be the same as, or different from, each other (or two can be the same as each other with the other being different). Each of the first temporal frequency, the second temporal frequency, and the third temporal frequency can be in a range of, for example, from 0.1 Hz to 100 Hz (e.g., 0.5 Hz to 100 Hz, 0.5 Hz to 10 Hz, or 1 Hz to 2 Hz (such as 1 Hz)). The method can further comprise: providing, during the acquiring of the spatio-temporal diffuse reflected maps, an external stimulus configured to alter peripheral tissue oxygenated flow under skin of the subject; analyzing the dynamic maps; generating flow correlation maps from the dynamic maps (e.g., obtained from a hemoglobin parameter or its related derivative(s), from the dynamic maps; and/or analyzing the spatio-temporal diffuse reflected maps. The method can further comprise: determining a likelihood that the subject has vascular calcification (VC) based on at least one of the following: the HbT map (if present); an extent of change in a hemoglobin parameter, or its related derivative(s), from the dynamic maps; the spatio-temporal diffuse reflected maps; a rate of occlusion or a slope of a hemoglobin parameter, or its related derivative(s), from the dynamic maps; and the flow correlation maps. Changes in parameters of the dynamic maps can be independent (or predominantly independent) of the color (and/or melanin concentration) of the skin of the subject (making the subject methods applicable for all different races and ethnic groups).

### BRIEF DESCRIPTION OF DRAWINGS

FIG. 1A shows a plot of pressure (in millimeters of mercury (mmHg)) versus time (in seconds(s)) for a vascular occlusion protocol used for imaging a mouse tail. At a time of 60 s (t=60s), the cuff on the mouse tail applied pressure until reaching a peak of 250 mmHg at t=72s. Occlusion pressure was maintained for 3s, then steadily released until t=90s. The occlusion cycle started after a 30 s rest period. A total of seven occlusion cycles were performed and imaged.

FIG. 1B shows a sample average hemoglobin-based concentration map of the mouse tail from FIG. 1A. The map was used to identify the blood vessel (the region at the bottom-left near the inset pixel) during the dynamic imaging session. The region of interest (ROI) was the selected area (4×4 pixels; the inset in the figure) within the tail's blood vessel. Three different ROIs were chosen within the mouse's tail vessel and its average and standard deviation was determined.

FIG. 2A shows a near-infrared optical scanner (NIROS) image field of view with the mouse tail, occlusion cuff, and fiducial marker.

FIG. 2B shows a mean total hemoglobin (HbT) spatial map of mouse tails derived from a modified Beer-Lambert's law. An ROI was chosen on the vessel, determined by color bar intensity.

FIG. 2C shows an average of HbT trendlines for the chosen ROI with respect to the time with mean error. This is presented in a plot of intensity (abu) versus time (in s).

FIG. 2D shows a normalization of HbT with respect to 60 s prior to the start of the first occlusion. This is presented in a plot of change in HbT (in percentage (%)) versus time (in s).

FIG. 2E shows a calculation of two parameters during the first occlusion cycle for comparison: extent of change; and occlusion slope. This is presented in a plot of change in HbT (in %) versus time (in s). Collectively, FIGS. 2A-2E form a flowchart of effective HbT data with respect to time. Analysis was completed for each mouse for the respective imaging session. 15

FIG. 3A shows a plot of change in parameter (in %) versus time (in s), showing oxygenation trends in response to an occlusion stimulus on a mouse model. The curve with the highest change in parameter value at 70 s is for change in oxy-hemoglobin (HbO) (ΔHbO); the curve with the second-highest change in parameter value at 70 s is for change in HbT (ΔHbT); the curve with the third-highest change in parameter value at 70 s is for change in deoxy-hemoglobin 20 (HbR) (ΔHbR); and the curve with the lowest change in parameter value at 70 s is for change in oxygen saturation (StO₂) (ΔStO₂). The trendlines were extracted from the selected ROI, of a sample mouse prior to the onset of calcification (i.e., week 6). Rest period for the mouse took place for the first 60 s (baseline), and the occlusion stimulus with a tail cuff began at 60 s. Initial 25 (green) shading represents the increase in pressure (i.e., 10 s), followed by (blue) shading (at 70-75 s) where max pressure of 250 mmHg is maintained (i.e., 5 s). The next (green) shading (at 75-90 s) is the release of pressure until 0 mmHg (i.e., for 15 s) is exerted, followed by rest (i.e., 30 s).

FIG. 3B shows plots of change in parameter (in %) versus time (in s) for HbO, HbR, HbT, and StO₂ (from top plot to bottom plot), showing average tissue oxygenation trendlines in terms of ΔHbO, ΔHbR, ΔHbT, and ΔStO₂ across all five mice during week 6 (prior to calcification) and week 12 (onset of calcification). Rest period for the mouse took place for the first 60 s, and the occlusion cycles on the tail began immediately after. The shaded region relates to the standard error across different mice at both timepoints (week 6 and week 12). In each plot, the curve with the greater change in parameter values is for week 6, and the curve with the lower change in parameter values is for week 12.

FIG. 4A shows a plot of change in parameter (in %) versus time (in s), showing typical total vascular occlusion induced oxygenation change in terms of HbO, HbR, and HbT as observed from prior studies (see also Irwin et al., Near infra-red spectroscopy: a non-invasive monitor of perfusion and oxygenation within the microcirculation of limbs and flaps, British Journal of Plastic Surgery 48:14-22, 1995; which is hereby incorporated by reference herein in its entirety). [The 150-sec mark was used to help distinguish the curves, as the figures will be in grayscale at the Patent Office. Even though it's not marked on the x-axis, it's apparent where the 150-sec mark would be. However, I removed this because the curves are labeled around that same point anyway.]

FIG. 4B shows an enlarged version of the portion of FIG. 4A marked with the dotted box. The curve with the highest change in parameter value at the far-right of the plot is for HbR; the curve with the second-highest change in parameter value at the far-right of the plot is for HbT; and the curve with the lowest change in parameter value at the far-right of the plot is for HbO. These zoomed-in trendlines within the first few seconds of occlusion onset demonstrate the immediate increase in HbT.

FIG. 5A shows a plot of change in HbT (ΔHbT (in %)) versus time (in s), showing HbT trendline normalized with respect to rest period of a mouse at timepoint 60 s prior to occlusion. Occlusion stimulus was applied beginning at the dashed divider. The mouse for FIG. 5A was a control mouse with induced chronic kidney disease (CKD) across weeks 6, 9, and 12.

FIG. 5B shows a plot of ΔHbT (in %) versus time (in s), showing HbT trendline normalized with respect to rest period of a mouse at timepoint 60 s prior to occlusion. Occlusion stimulus was applied beginning at the dashed divider. The mouse for FIG. 5B was a disease model sample mouse as the CKD-induced with a vascular calcification (VC) from a high phosphate (HP) diet (i.e., CKD+VC mice model (also referred to as CKD+HP mice model)) presence across weeks 6, 9, and 12.

FIG. 6A shows a bar chart for ΔHbT across a group of six control mice (included CKD), showing the extent of change during occlusion cycle of HbT. For each control mouse, the left bar is for week 6, the middle bar is for week 9, and the right bar is for week 12.

FIG. 6B shows a bar chart for ΔHbT across a group of five test mice (CKD+HP), showing the extent of change during occlusion cycle of HbT. For each control mouse, the left bar is for week 6, the middle bar is for week 9, and the right bar is for week 12. With respect to both FIG. 6A and FIG. 6B, each week was individually normalized to the mouse at rest, anesthetized with 2% isoflurane. The extent of change in total hemoglobin being a response to the first occlusion cycle on the mouse tail was calculated by a difference between the maximum and minimum. Data is presented as the three-region average±standard error for each individual mouse.

FIG. 7A shows a bar chart for ΔHbT for the control mice, showing a grand mean±standard error comparison of the extent of change in total hemoglobin in response to an occlusion stimulus. The left bar is for week 6, the middle bar is for week 9, and the right bar is for week 12.

FIG. 7B shows a bar chart for ΔHbT for the test mice, showing a grand mean±standard error comparison of the extent of change in total hemoglobin in response to an occlusion stimulus. The left bar is for week 6, the middle bar is for week 9, and the right bar is for week 12.

FIG. 8A shows a bar chart for slope (occlusion rate of HbT per mouse) for the control mice. For each control mouse, the left bar is for week 6, the middle bar is for week 9, and the right bar is for week 12.

FIG. 8B shows a bar chart for slope (occlusion rate of HbT per mouse) for the test mice. For each test mouse, the left bar is for week 6, the middle bar is for week 9, and the right bar is for week 12. With respect to both FIG. 8A and FIG. 8B, each week was individually normalized to the mouse at rest, anesthetized with 2% isoflurane. The occlusion rate of change in total hemoglobin (ΔHbT) being a response to the first occlusion cycle on the mouse tail was calculated by linear fit of the first five seconds. Data is presented as the three-region average±standard error for each individual mouse.

FIG. 9A shows a bar chart for slope (occlusion rate of HbT per mouse) for the control mice, showing a grand mean±standard error comparison of the extent of change in total hemoglobin in response to an occlusion stimulus. The left bar is for week 6, the middle bar is for week 9, and the right bar is for week 12.

FIG. 9B shows a bar chart for slope (occlusion rate of HbT per mouse) for the test mice, showing a grand mean±standard error comparison of the extent of change in total hemoglobin in response to an occlusion stimulus. The left bar is for week 6, the middle bar is for week 9, and the right bar is for week 12.

### DETAILED DESCRIPTION

Embodiments of the subject invention provide novel and advantageous systems, devices, and methods for performing peripheral vascular imaging (e.g., in a mammal, such as a human or a mouse). A continuous wave, non-contact, near-infrared optical scanner (NIROS) can utilize at least one near-infrared (NIR) light (e.g., light-emitting diode (LED), laser diode, or any other suitable NIR light source) (such as multi-wavelength (e.g., dual-wavelength, tri-wavelength, or more) NIR lights (e.g., LEDs)). The diffuse reflected NIR signal(s) can be obtained from the surface of the tissue of the mammal being imaged. The signal(s) can be optically filtered (e.g., through a long pass filter) and can be detected by an NIR-sensitive image sensor (e.g., a camera, such as a complementary metal oxide semiconductor (CMOS) camera, charge coupled device (CCD) camera, or any other suitable NIR-sensitive image-sensor). A graphical user interface (GUI) can be used to automate the acquisition of the (multi-wavelength) spatio-temporal diffuse reflected maps from the NIROS device.

Measurement of vascular calcification (VC) typically requires large, expensive imaging modalities such as computed tomography or magnetic resonance angiography and is often focused clinically on large arterial beds. The relationship between cardiothoracic and peripheral calcification is unclear. This limits the diagnostic utility for underserved populations in low-resource settings. Further, therapeutics to prevent or inhibit VC would be beneficial, but diagnostic strategies are needed to identify appropriate patients and monitor therapeutic efficacy. Low-cost, non-invasive techniques of embodiments of the subject invention to assess VC (e.g., in individuals with chronic kidney disease (CKD)) can enhance risk stratification and enable the advent of therapies to improve cardiovascular health. It is expected that stiffened, calcified vasculature is less compliant; therefore, total hemoglobin within the vessel will exhibit a distinct difference during constriction and reperfusion.

Embodiments of the subject invention can utilize a low-cost, non-contact, NIROS that can provide spatio-temporal based tissue oxygenation mapping and perfusion in tissues. Unlike LDPM that maps dynamic changes in perfusion (or blood flow at a single point location), the NIRS-based approach implemented by a NIROS can obtain these near-real-time tissue oxygenation-based perfusion maps across a wide-field region of the imaged peripheries. Embodiments of the subject invention can measure dynamically changing two-dimensional (2D) spatial tissue oxygenation maps (in terms of hemoglobin concentration parameters or their related derivatives) at the peripheries. Imaging across the entire peripheral region thus reduces subjectivity when clinically applied. These near real-time spatio-temporal maps of tissue oxygenation changes in the peripheries can map vascular response to underlying peripheral issues upon introducing a stimulus to induce vascular changes.

NIROS devices of embodiments of the subject invention can map for spatio-temporal changes in response to a stimulus that alters the peripheral tissue oxygenated flow under the skin. The NIROS devices can also perform peripheral vascular imaging and identify tissue oxygenation changes in a mammal (e.g., a human or a mouse), either with or without VC (e.g., induced VC).

Embodiments of the subject invention provide non-contact, NIR optical imaging devices, which can be hand-held (i.e., standalone hand-held), smartphone-based, or mobile-based), and can perform spatio-temporal NIR imaging. Such imaging can be performed on, for example, the peripheries (i.e. peripheral limbs and/or tail (if present)) of the subject (e.g., a mammalian subject).

The NIR imaging techniques can use a stimulus (e.g., auto-stimulus or induced stimulus) to cause dynamic changes in the single- or multi-wavelength diffuse reflected signals and/or hemodynamic signals (e.g., in terms of oxy-hemoglobin (HbO), deoxy-hemoglobin (HbR), total hemoglobin (HbT), and/or oxygen saturation (StO₂) or its respective derivative parameters) to determine extent of change, rate of change, volume of change, and/or flow pattern changes beneath the surface of the skin of the subject (e.g., the skin in the peripheries). This can be used to, for example, detect the presence, absence, and/or onset of VC (e.g., peripheral VC).

A vascular occlusion (e.g., venous, arterial, and/or total occlusion based) induced stimulus can be used to induce vasoconstriction/vasodilation to observe changes in one or all of the parameters (HbO, HbR, HbT, StO₂, and/or any of the respective derivative parameters). The vascular occlusion induced stimulus can have a timing of pre-occlusion, during occlusion, or post-occlusion timings. Any other stimulus (e.g., breath-hold, occlusion, temperature induced, or other stimulus) that can observe changes in all above parameters in tissue oxygenation or the detected NIR signals, and/or flow pattern changes across any skin color (or Fitzpatrick scale) without being limited by the skin color (light versus dark) can be used.

Image analysis techniques of embodiments of the subject invention can measure intensity-independent measurements of time-varying diffuse reflectance and/or hemodynamic signals, allowing imaging across spatially varying skin colorations (of varying melanin concentrations) of tissues. Imaging and analysis techniques can develop a VC-related index that determines the presence/absence and/or onset of VC and/or peripheral calcifications based on intensity-independent diffuse reflectance and/or hemodynamic signals, as a new indicator of diseased state of the tissues (e.g., detect calcifications). The imaging and analysis techniques are also less impacted (compared to prior art techniques) or not impacted at all by spatially varying skin colorations or pigmentations (i.e. melanin concentrations).

Embodiments of the subject invention provide application software (e.g., instructions stored on a machine-readable medium that, when executed by a processor, perform steps or actions) for spatio-temporal data acquisition of the multi-wavelength diffuse reflectance signals (NIR wavelengths) to develop a calcification indicator based on differences in diffuse reflectance signals, tissue oxygenation signals (in terms of HbO, HbR, HbT, StO₂, and/or related derivatives), extent of change of these parameters during perfusion, reperfusion, and/or occlusion state, rate of perfusion, reperfusion and/or occlusion state, and differences in flow patterns during perfusion, reperfusion, and/or occlusion state. The application software can be synced with the NIR optical imaging device (e.g., via wireless (such as Bluetooth) or wired technology). The software can provide data pre-processing and analysis from one or more of the dynamic images (single or multi-wavelength NIR images at specific wavelengths of choice), which are displayed individually or coregistered onto each other. The software can process the multi-wavelength diffuse reflectance images to generate dynamic maps of the same images, tissue oxygenation maps (in terms of HbO, HbR. HbT, StO₂, and/or related derivatives), white light maps, and/or all parameters listed herein to obtain the calcification based indicator (or index). The software can perform coregistration and/or image segmentation of each or any of these images with respect to each other, or all images into a single coregistered and/or segmented image. Segmentation can be manual, or using artificial intelligence (e.g., deep learning, machine learning, and/or convolution neural networks).

Embodiments of the subject invention provide a novel time-varying diffuse reflectance-based indicator, which can be independent of spatially varying skin tones when imaging various disease models (e.g., detect calcifications). The time-varying diffuse reflectance-based indicator can assess the presence or absence, and/or onset, of vascular and/or peripheral calcification or diseased tissues. Embodiments also provide a novel time-varying tissue oxygenation-based and/or flow-based indicator that is independent of spatially varying skin tones when imaging various disease models (e.g., detect calcifications). The time-varying tissue oxygenation-based and/or flow-based indicator can also assess the presence or absence, and/or onset, of vascular and/or peripheral calcification or diseased tissues.

In an embodiment, a continuous wave, non-contact NIROS can be used to perform vascular imaging (e.g., peripheral vascular imaging). The NIROS can utilize multi-wavelength NIR LEDS in the 650 nanometer (nm)-950 nm range (e.g., 690 nm and 830 nm for dual-wavelength). Each wavelength can have optical power (e.g., 32 milliwatts (mW) of optical power) at the source end and can be multiplexed at a temporal frequency (e.g., 2 Hertz (Hz); equivalent to 1 Hz per wavelength) via an LED driver. The diffuse reflected NIR signals can be obtained at both the wavelengths from the tissue surface. These signals can be optically filtered through a filter (e.g., a long pass filter such as a 645-nm long pass filter) before being detected by an NIR-sensitive image sensor (e.g., a camera such as a CMOS camera). A GUI (e.g., a MATLAB-based GUI) can be used to automate the acquisition of the (multi-wavelength (e.g., dual-wavelength)) spatio-temporal diffuse reflected maps from the NIROS device. The GUI can be stored on a machine-readable medium in operable communication (e.g., wireless or wired) with the NIROS device.

Embodiments of the subject invention can be used for, e.g., detecting peripheral vascular disease, peripheral vascular calcification, vascular calcification with or without chronic kidney disease, and/or peripheral arterial disease.

Embodiments of the subject invention haver certain aspects in common with U.S. Pat. Nos. 10,674,916, 11,464,453, and 11,471,696, all of which are hereby incorporated by reference herein in their entireties.

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

### Materials and Methods

A continuous wave, non-contact, NIROS was used to perform peripheral vascular imaging. The NIROS utilized dual-wavelength NIR LEDs in the 650 nm-950 nm range. Each wavelength (682 nm and 826 nm) had 32 milliwatts (mW) of optical power at the source end and were multiplexed at 2 Hertz (Hz) temporal frequency (equivalent to 1 Hz per wavelength) via a custom LED driver. The diffuse reflected NIR signals were obtained at both the wavelengths from the tissue surface. These signals were optically filtered through a 645 nm long pass filter (LP 645, MidOpt) before being detected by an NIR-sensitive CMOS camera (IDS, Germany). A MATLAB-based GUI was used to automate the acquisition of the (dual-wavelength) spatio-temporal diffuse reflected maps from the NIROS device.

A peripheral vascular feasibility imaging study was performed on mice with vascular calcification induced via a high phosphate diet. In this study, CKD was induced in adult mice by feeding them a high adenine diet (0.2%) for 6 weeks. At week 6, the high-adenine diet was supplemented with high phosphate (1.8%) to also induce VC. A peripheral vascular occlusion test was carried out before (week 6) and after (week 12) the addition of the high phosphate to the adenine diet, and the mice were imaged using the NIROS. Differences in hemoglobin concentration maps in response to peripheral occlusion were compared before and after the onset of vascular calcification.

At 10 weeks of age, ten C57BL/6J mice were placed on a 0.2% adenine diet for six weeks to induce CKD. For the remaining 6 weeks of the study, the diet was supplemented with 1.8% phosphate to induce VC (CKD+HP), a pathological feature common in CKD-induced vascular dysfunction. Mouse weight was monitored and recorded throughout the study and considered in analyses. Three mice died prior to week 12, and two mice had data acquisition errors. Hence, only five mice were assessed in the current study on peripheral vascular imaging in response to VC (Example 1).

In Example 2 and Example 3, there were two groups of mice. A CKD group was kept on the 0.2% adenine diet. A CKD with high phosphate group (CKD+HP or termed CKD+VC to indicate VC) was supplemented at week 6 with 1.8% phosphate diet to induce VC. A total of six mice in each group were imaged week 6, week 9, and week 12, and the changes in tissue oxygenation and rate of perfusion were compared within and across the two groups.

The NIROS was used to perform peripheral vascular imaging of the mouse tails (dorsal side) in response to an occlusion protocol. The device acquired spatio-temporal maps at week 6 (prior to adding high phosphate and without the presence of VC) and at week 12 of the diet. The mice were placed in a prone position, temperature controlled, and anesthetized using isoflurane throughout data acquisition. Imaging was carried out at 2 Hz frequency by multiplexing between the two source wavelengths (682 nm and 826 nm) for 10 minutes. For the first 5 minutes, mice were imaged while their body temperature stabilized and without applying any occlusion. During the following 5 minutes, a tail-cuff (at the base of the tail) was used to occlude the tail. A systematic occlusion cycle was generated using a Kent CODA blood pressure monitor device.

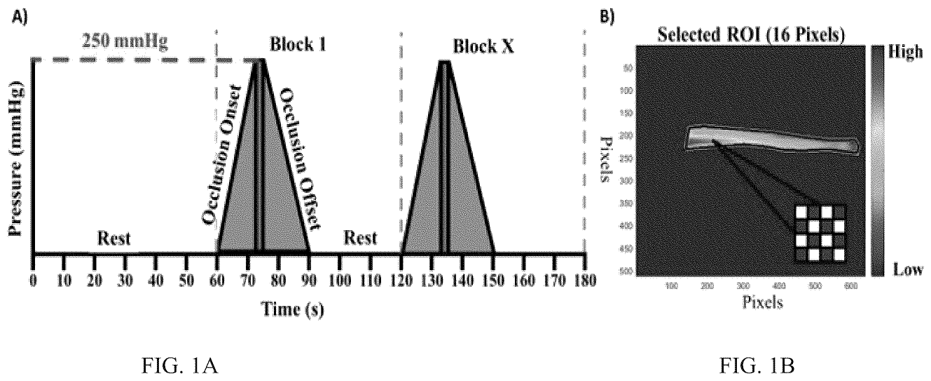

The tail was occluded seven times using a 60-second stimulus cycle (12 seconds(s) from rest to full occlusion, 3 s occlusion, 15 s for deflation, and 30 s for rest) (see also FIG. 1A). The dynamically changing diffuse reflectance NIR measurements (at both the source wavelengths) were obtained in response to the occlusion cycles.

A diffusely reflective calibration sheet was used prior to each imaging session to obtain the reference NIR signals. The effective hemoglobin-based concentration maps (in terms of [ΔHbO], [ΔHbR], [ΔHbT], and [ΔStO₂]) were calculated using modified Beer-Lambert's Law (MBLL) on the time-varying diffusely reflected NIR maps. There were no motion artifacts; hence, image coregistration across the time-varying signal was not performed. Changes in the effective hemoglobin parameters with respect to time were determined at a selected region of interest (ROI) (FIG. 1B). The ROI (4×4 pixels) was chosen on the tail proximal to the body, and the 16-pixel average was considered as the trend for the area. Three ROIs were chosen on the tail's blood vessel for each mouse and averaged to determine the variability of the measured oxygen saturation (with each mouse).

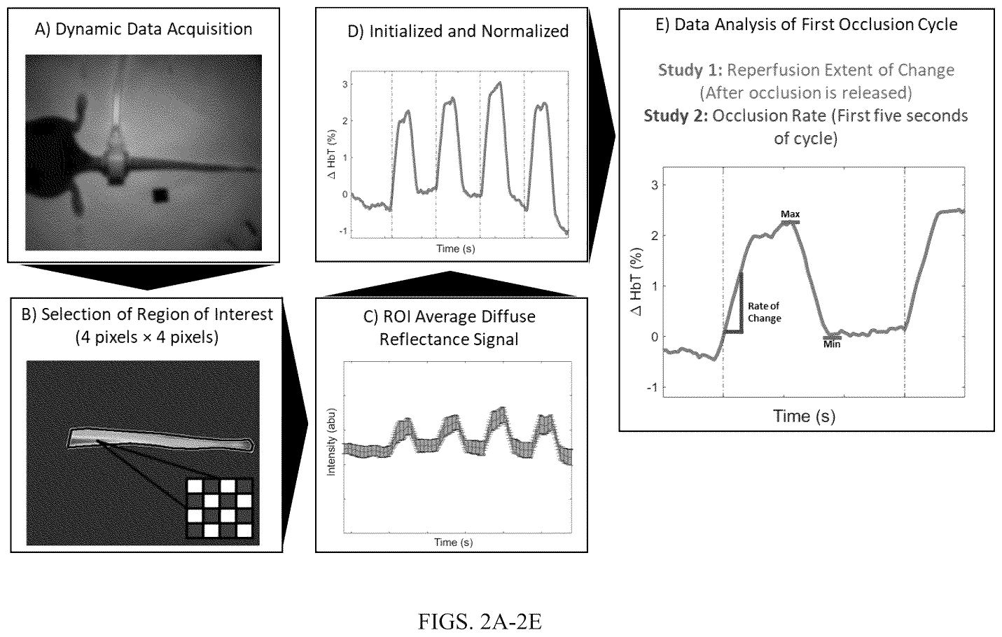

The trendline for each pixel within the ROI (16 pixels total) was utilized to calculate the average trendline for vessel location (FIG. 2C). The effective HbT was calculated by the summation of the effective concentration of HbO and HbR within the chosen ROI before further calculations.

Synchronization of the spatio-temporal maps was completed with the occlusion start time which was annotated during each imaging session. Because the mice vary in size and biological sex, HbT was initialized and normalized with respect to the rest timepoint (HbT₀), 60 seconds prior to first occlusion cycle (FIG. 2D). Three ROIs were chosen per imaging session to evaluate the variance of the subjective selection. All subsequent analysis was based on the selected ROIs.

### Example 1—Feasibility Study of Peripheral Vascular Imaging in Mice with and without VC

Hemoglobin-based parameters were normalized by calculating the percentage of change with reference to the respective parameters 60 seconds prior to the start of the occlusion cycles. The time-synchronized trendlines for the oxygenation parameters were compared for a qualitative assessment of the blood flow without the presence of calcification (week 6). The evaluation of all parameter responses analogized the occlusion on a mouse model with a human. Further analysis included the comparison of the absence of calcification (week 6) and the presence of calcification (week 12). With the week 6 and 12 trendlines, the extent of change was quantified per parameter. The extent of change was calculated by the difference in the percentage change when the occlusion pressure reached its peak (250 mmHg) and the plateau value that followed (0 mmHg).

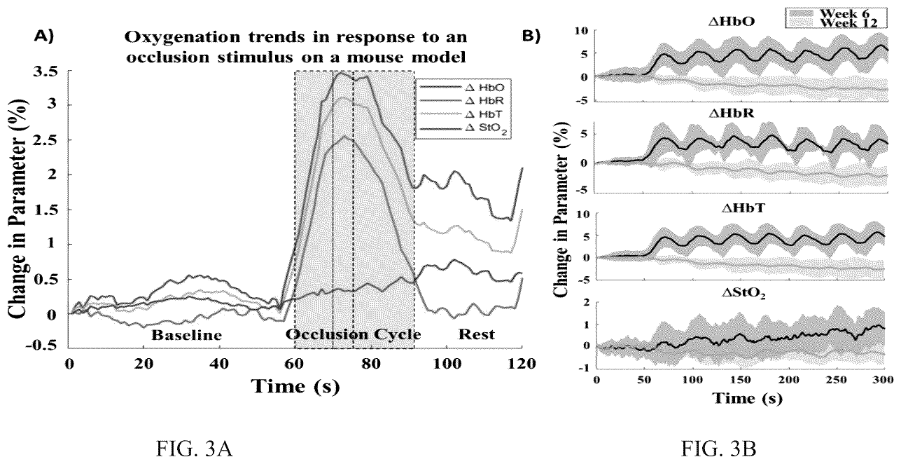

The changes in tissue oxygenation parameters (in terms of ΔHbO, ΔHbR, ΔHbT, and ΔStO₂) across the rest period and the first occlusion cycle from one sample case of the mouse model prior to vascular calcification (i.e., at week 6) is shown in FIG. 3A. A distinct increase in ΔHbO, ΔHbR, and ΔHbT with the onset of occlusion was observed. These three parameters decreased at the pressure release point; however, ΔHbR was the only one to return to baseline. ΔStO₂ slightly increased, but in comparison to the other parameters, it was least impacted from occlusion, possibly because the mouse tail did not reach ischemic state with the short occlusion cycle.

Extracted tissue oxygenation parameters were compared between week 6 (prior to vascular calcification) and week 12 (with the onset of vascular calcification) groups, as shown in FIG. 3B. The changes in ΔHbO, ΔHbR, and ΔHbT were distinct in response to occlusion at week 6. However, this response was vaguely seen in the mice upon onset of calcification (at week 12). The occlusion protocol was maintained the same for both week 6 and week 12. Hence, the lack of response to the occlusion in week 12 is possibly due to the presence of vascular calcification. The variability (or standard error) among the cohort as observed distinctly in week 6 is possibly due to differences in body weight and biological sex of these mice. Despite the variance amongst mice, similar waveform changes was observed across the mice during both week 6 and week 12. This feasibility study on five mice clearly distinguished the two mice groups (at week 6 prior to and week 12 with onset of calcification).

Cardiovascular disease is the leading cause of death in individuals with CKD, and VC is the best predictor of CKD-associated cardiovascular morbidity. Individuals with CKD also exhibit peripheral vascular dysfunction, and VC can occur in peripheral arteries. Even without mineral deposition, calcification in larger arterial beds may alter blood flow patterns and perfusion throughout systemic circulation. Low-cost techniques that assess peripheral vascular dysfunction could potentially replace expensive imaging techniques (such as computed tomography or magnetic resonance angiography) for regular monitoring of cardiovascular health in CKD patients.

Low-cost, non-invasive techniques to assess the onset of VC via peripheral vascular imaging in human subjects, such as those of embodiments of the subject invention, can enhance risk stratification and enable the advent of therapies to improve cardiovascular health. The prior art has no teaching focused on assessing the microcirculatory changes in peripheral vascular tissue oxygenation in mice (or other mammals) before and after the onset of VC.

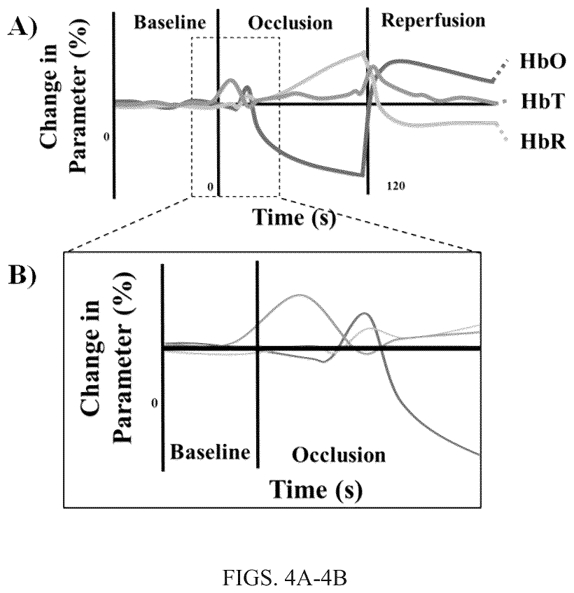

In this NIRS imaging example, prior to calcification (week 6), occlusion resulted in an initial increase in oxygenation parameters (ΔHbO, ΔHbR, and ΔHbT) as shown in FIGS. 3A and 3B. The tail occlusion in these mouse models was expected to cause a total occlusion (both arterials and veins) as the maximum occlusion pressure was 250 mmHg (well above arterial pressures). The changes in tissue oxygenation parameters typically follow the trend shown in FIGS. 4A and 4B. In this example (and Examples 2 and 3), it was observed that all the tissue oxygenation parameters increased upon occlusion, unlike a decrease in HbO as seen in the hind limbs of rabbits in the related art. This difference in the hemodynamic response could be from the 2-minute occlusion performed in the related art compared to a maximum of 5 seconds of occlusion in this example (and Examples 2 and 3). Interestingly, the HbT was shown to increase immediately upon the onset of occlusion (as shown in FIG. 4B), which is similar to what was observed within the 5-second occlusion as well.

The changes in ΔHbO, ΔHbR and ΔHbT were observed in response to occlusions in mice at both the time points (prior to and with onset of vascular calcification at week 6 and 12, respectively). However, the extent of change significantly diminished from week-6 to week-12 with the onset of vascular calcification. The extent of change in ΔHbT in response to occlusion (area under the curve) possibly relates to the total blood volume, similar to the direct relation of ΔHbT to the cerebral blood volume in brain imaging studies using NIRS. The drop in the ΔHbT response from week 6 to week 12 (with the onset of vascular calcification) can also imply a change in the total blood volume in the peripheries.

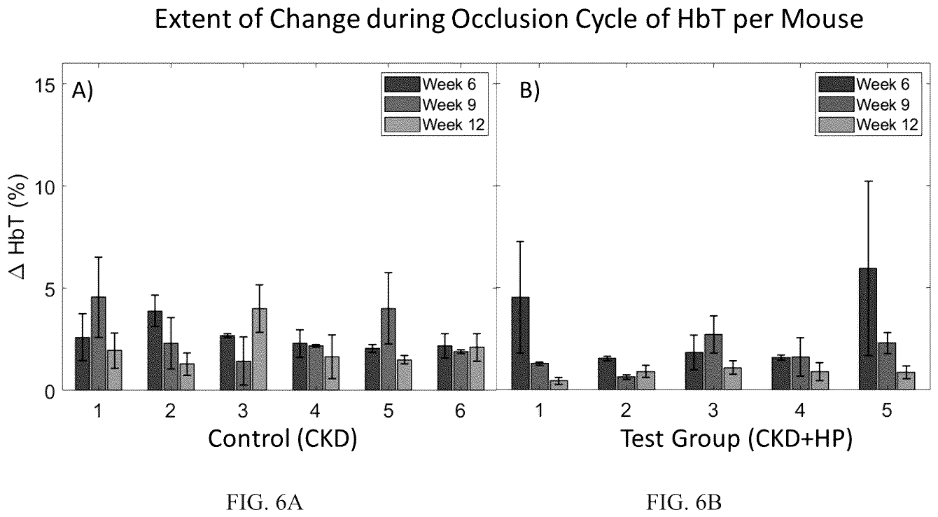

The ΔStO₂ in this example did not change significantly in response to the multiple occlusion cycles, indicating that the occlusion was not maintained for sufficient time to bring the tail to an ischemic state. The time the mice remained in the total occlusion state was about 5 s, which was possibly not sufficient for the tail to reach the complete ischemic state or even cause a significant change in oxygen saturation during each occlusion cycle. However, there was a slight overall increase in ΔStO₂ across the occlusion cycles as seen from FIGS. 6A and 6B.

There was no distinct drop in the blood pressure of these mice from week 6 to week 12. At week 6, the mice had the following blood pressure averages (+standard error of measurement (SEM)): mean arterial pressure of 78.00+2.97 mmHg, diastolic pressure 72.07+2.77, and systolic pressure of 91.13+3.58 mmHg. At week 12, the mice had the following blood pressure averages (+SEM): mean arterial pressure of 71.59+2.03 mmHg, diastolic pressure 67.98+1.93, and systolic pressure of 79.45+2.21 mmHg. This may imply that the blood pressures are not abnormally low during week 12 and are possibly not the cause for the decreased ΔHbT changes in week 12 from week 6. Similarly, there was no change in the average weight of the mice with a change in diet from week 6 to week 12. The average weight for both weeks was 18.87 grams (g), with week 6 having a range of 17.85 g-20.55 g and week 12 a range of 15.81 g-20.69 g. This implies that the decreased ΔHbT changes in week 12 with respect to week 6 is not from weight loss or gain with dict. Additionally, the heart rate of the mice used in this example was not recorded. Although the heart rate could influence tissue oxygenation, the mouse heart rate is very high (>300 beats per minute (bpm) or >5 Hz). In this example, the imaging was performed at 1 Hz frequency. Hence, the tissue oxygenation changes in response to the heart rate cannot be captured nor impact our current results due to the high frequency of the heart rate compared to the imaging frequency.

This example shows that the NIROS device can detect vascular remodeling-associated changes in hemodynamics. A mouse model of VC was used to identify whether the NIROS device can detect changes in oxygenation of peripheral vessels. The presence of VC in peripheral arteries increases mortality and limb-specific outcomes. Therefore, it is important to identify and monitor the presence of peripheral VC. Patients with CKD also exhibit microvascular dysfunction, and changes in microcirculation associate with VC in these patients. NIROS-based assessment of superficial hemodynamics can provide a non-invasive approach to gauge general vascular health in CKD patients.

Systemic circulation can be easily monitored using blood pressure cuffs. Although systemic blood pressure can change due to the presence of calcification, there are multiple conditions that can cause systemic changes in blood pressure (heredity, smoking, obesity, etc.). The NIROS device can be used to detect local changes in tissue oxygenation of peripheral vessels due to the presence of VC.

### Example 2—Extent of Tissue Oxygenation Change During Reperfusion

Using the processed dynamic data, the extent of change and the occlusion rate was calculated across all timepoints. Extent of change (ΔHbO, ΔHbR, ΔHbT) during the reperfusion (release of occlusion) was calculated using maximum and minimum peak values were constrained to the first occlusion cycle of the session for exclusions of possible insufficiencies caused by multiple occlusions. The extent of HbT change for the three regions of interest was extracted and then averaged to represent the mouse response (FIG. 2E). Variance of subjective ROI selection point was determined by the standard error of the three regions. Successively, the grand mean was calculated based on each three-region mouse average. Grand mean comparison of both groups was used to analyze changes between week 6 and week 12 of HbT as bar plot graphic with the mean and standard error per group.

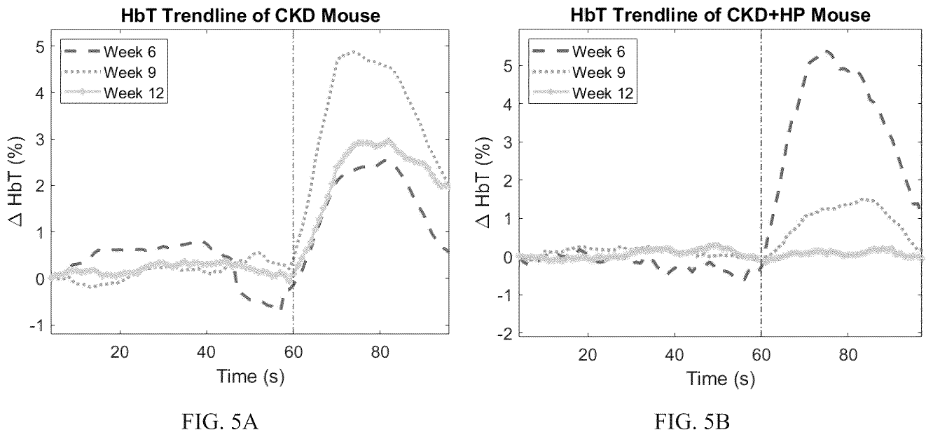

Trendlines for the effective oxygenation parameters were normalized (with respect to 60 s prior to occlusion) and then plotted per mouse to compare the changes across weeks. A CKD mouse and a CKD+HP (also referred to as CKD+VC) mouse were compared as the diseases progressed, as seen in FIGS. 5A and 5B. The first 60 s represent the temperature-stabilized time prior to occlusion, while the following 30 s include the first occlusion cycle. The ΔHbO, ΔHbR, and ΔHbT correlated with a response to the occlusion stimulus from the Kent CODA device. A qualitative comparison between week 6 and week 12 of the CKD mouse trendline showed no distinct change in the extent of ΔHbT. In contrast, the CKD+HP mouse trendline at week 12 showed no response to the occlusion stimulus, hence, a distinct change in the extent of ΔHbT. Because both mice groups were on the same diet at week 6, the differentiation at week 12 was quantified to further examine the effect of arterial calcification due to CKD.

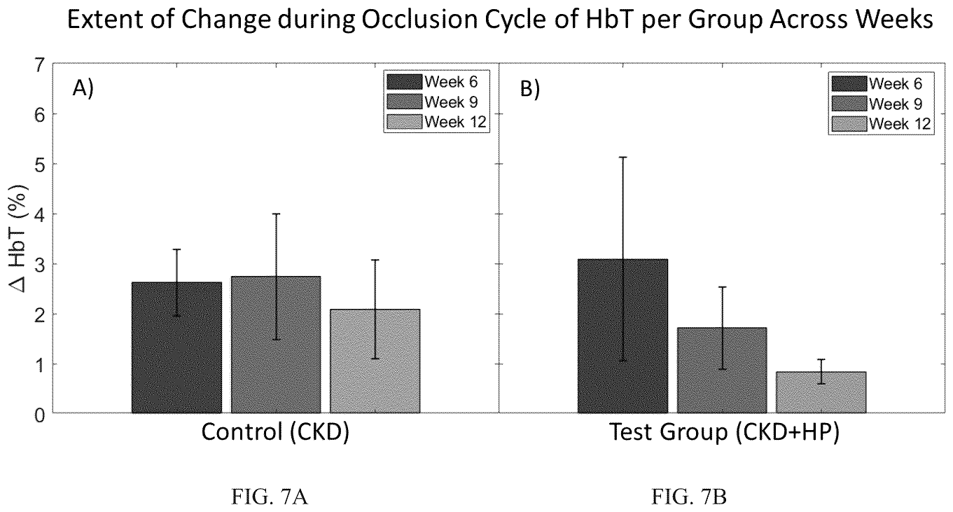

A quantitative assessment of the extent of ΔHbO, ΔHbR, and ΔHbT was completed for each mouse per each imaging session. Bar plots presented the three-region mouse average with the standard error across the imaging sessions, as seen in FIGS. 6A and 6B. Individual evaluation of the CKD group appeared to have no distinct relationship across the weeks. Five out of the six CKD+HP mice had a reduction in the extent of oxygenation from week 6 to week 12. A distinct change was seen for all the parameters. The individual assessment of each mouse at each imaging session permitted the identification of possible outliers. The grand mean calculated from the three-ROI average per mouse was used to compare the disease groups, as seen in FIGS. 7A and 7B. The CKD group maintained a steady ΔHbO, ΔHbR, and ΔHbT from week 6 to week 12, while the CKD+VC had a distinct change with the group.

### Example 3—Rate of Perfusion During Occlusion (Occlusion Slope)

With the same processed dynamic data from Example 1 (only the first occlusion cycle), the rate of change during the occlusion was calculated. The time it takes for the KENT CODA monitor to occlude was found to be 10-12 seconds, so the desaturation slope was considered for the first 6 seconds (FIG. 2E). The occlusion slope for the three regions of interest was extracted and then averaged to represent the mouse response. In continuation, the grand mean±standard error was calculated based on each three-region mouse average, as done in Example 2.

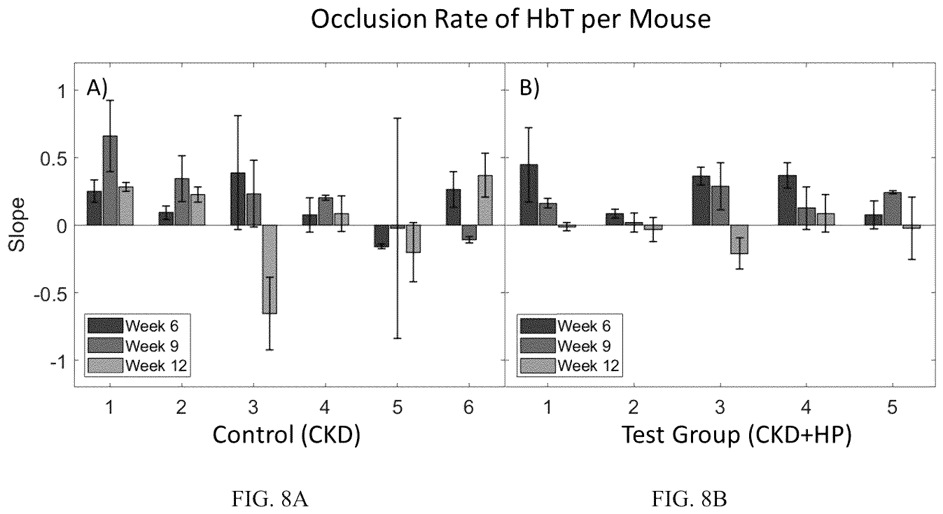

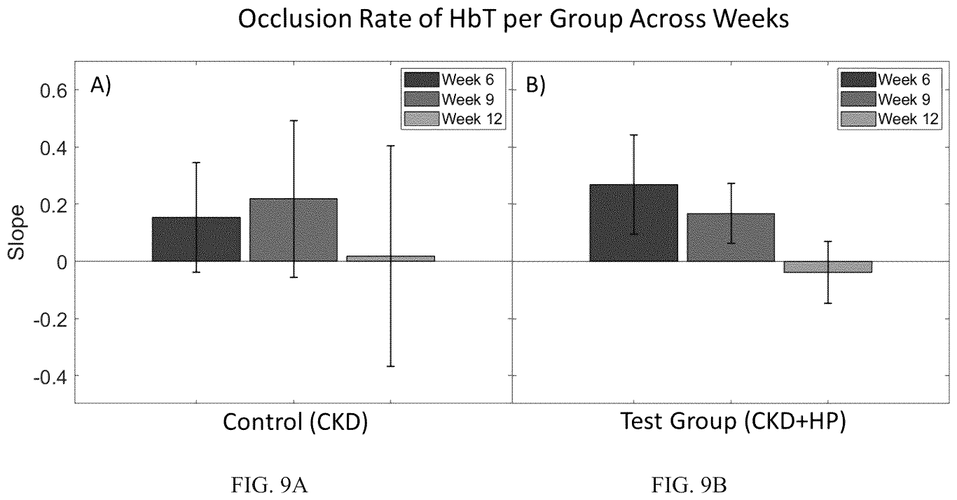

A second quantitative assessment of the oxygenation trendlines was conducted to compare the change in the perfusion rate in response to occlusion of the mouse tail. With a linear regression from the initiation of the occlusion cycle until half the time to full occlusion (6 s), the rate of change was calculated, and shown for each mouse across the groups (FIGS. 8A and 8B) and grand average across the mice in each group (FIGS. 9A and 9B), similar to that in Example 2.

From Example 2 and Example 3 across the two mice groups (CKD and CKD+VC or CKD+HP), it was observed that with vascular calcification onset by week 12, the extent of change in HbT during reperfusion (i.e., release from occlusion) significantly decreased from week 6 to week 12 in the CKD+VC mice. Similarly, the rate of perfusion during occlusion was also observed to have significantly dropped from week 6 to week 12 in the CKD+VC mice.

The time-varying tissue oxygenation parameters (in terms of HbO, HbR, HbT, StO₂, and/or related hemoglobin-based parameters) can also be correlated spatially in the peripheries to obtain flow correlation maps. These flow correlation maps (or flow-based indicator(s)) can in turn be used to determine the synchrony or asynchrony in the flow in response to occlusion or similar stimulus in the microcirculatory responses of the peripheries, or differences in response to onset of any disease model (e.g., CKD, VC, peripheral calcification etc.). The flow correlation maps can also be generated from the spatio-temporal diffusely reflected NIR signals obtained at different NIR wavelengths. The correlation maps can be used along with the extent of change and/or rate of change in tissue oxygenation parameters during occlusion, re-perfusion (i.e. post-occlusion), and/or rest periods during the occlusion paradigm.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

1. A method for performing non-contact, peripheral vascular imaging on a subject, the method comprising:
providing a near-infrared (NIR) optical imager comprising at least one NIR light, a filter configured to optically filter ambient light and allow only NIR light to pass, and an NIR-sensitive image sensor configured to detect NIR signals reflected from tissue of the subject;
applying a vascular occlusion protocol to the subject;
utilizing the NIR optical imager to scan tissue of the subject in a non-contact manner both before the vascular occlusion protocol is applied and while the vascular the vascular occlusion protocol is applied;
acquiring spatio-temporal diffuse reflected maps based on the reflected NIR signals detected by the NIR-sensitive image sensor;
generating dynamic maps based on the spatio-temporal diffuse reflected maps, the dynamic maps comprising at least one of a total hemoglobin (HbT) map, an oxy-hemoglobin (HbO) map, a deoxy-hemoglobin (HbR) map, and an oxygen saturation (StO₂) map for a region of interest (ROI) of the tissue of the subject;
displaying, via a graphical user interface (GUI) stored on a machine-readable medium in operable communication with the NIR optical imager, the dynamic maps;
analyzing the dynamic maps; and
determining, based on the dynamic maps, a likelihood that the subject has vascular calcification (VC) in the heart,
the filter being a long-pass filter or a band-pass filter.

2. (canceled)

3. The method according to claim 1, the subject being a human subject.

4. (canceled)

5. The method according to claim 1, the at least one NIR light of the NIR optical imager being a multi-wavelength light.

6. The method according to claim 5, the multi-wavelength light being a dual-wavelength light configured to emit light at a first wavelength and a second wavelength different from the first wavelength, and
each of the first wavelength and the second wavelength being in a range of from 650 nanometers (nm) to 950 nm.

7. The method according to claim 6, the at least one NIR light of the NIR optical imager being a light-emitting diode (LED),
the NIR optical imager further comprising an LED driver configured to multiplex light from the at least one NIR light, and
the method further comprising multiplexing the first wavelength and the second wavelength at a first temporal frequency and a second temporal frequency, respectively.

8. The method according to claim 7, the first temporal frequency being the same as the second temporal frequency.

9. The method according to claim 8, the first temporal frequency being in a range of from 0.5 Hertz (Hz) to 100 Hz.

10. The method according to claim 5, the multi-wavelength light being configured to emit light at a first wavelength, a second wavelength different from the first wavelength, and a third wavelength different from the first wavelength and the second wavelength, and
each of the first wavelength, the second wavelength, and the third wavelength being in a range of from 650 nanometers (nm) to 950 nm.

11. The method according to claim 10, the at least one NIR light of the NIR optical imager being a light-emitting diode (LED),
the NIR optical imager further comprising an LED driver configured to multiplex light from the at least one NIR light, and
the method further comprising multiplexing the first wavelength, the second wavelength, and the third wavelength at a first temporal frequency, a second temporal frequency, and a third temporal frequency, respectively.

12. The method according to claim 11, the first temporal frequency being the same as both the second temporal frequency and the third temporal frequency.

13. The method according to claim 12, the first temporal frequency being in a range of from 0.5 Hz to 100 Hz.

14. The method according to claim 2, further comprising:
analyzing the dynamic maps; and
determining the likelihood that the subject has VC in the heart based on an extent of change in a hemoglobin parameter, or its related derivative, from the dynamic maps.

15. The method according to claim 1, further comprising:
analyzing the spatio-temporal diffuse reflected maps; and
determining the likelihood that the subject has VC in the heart based on the spatio-temporal diffuse reflected maps.

16. The method according to claim 2, further comprising:
analyzing the dynamic maps; and
determining the likelihood that the subject has VC in the heart based on a rate of occlusion of a hemoglobin parameter, or its related derivative, measured from the dynamic maps in rear-real-time.

17. The method according to claim 2, further comprising:
generating flow correlation maps from the dynamic maps; and
determining the likelihood that the subject has VC in the heart based on the flow correlation maps.

18. The method according to claim 2, the vascular occlusion protocol comprising providing an external stimulus configured to alter peripheral tissue oxygenated flow under skin of the subject.

19. The method according to claim 2, where changes in parameters of the dynamic maps are independent of a color of skin of the subject.

20. A method for performing non-contact, peripheral vascular imaging on a subject, the method comprising:
providing a near-infrared (NIR) optical imager comprising at least one NIR light, a filter configured to optically filter ambient light and allow only NIR light to pass, and an NIR-sensitive image sensor configured to detect NIR signals reflected from tissue of the subject;
applying a vascular occlusion protocol to the subject;
utilizing the NIR optical imager to scan tissue of the subject in a non-contact manner both before the vascular occlusion protocol is applied and while the vascular occlusion protocol is applied;
acquiring spatio-temporal diffuse reflected maps based on the reflected NIR signals detected by the NIR-sensitive image sensor;
generating dynamic maps based on the spatio-temporal diffuse reflected maps, the dynamic maps comprising an oxy-hemoglobin (HbO) map, a deoxy-hemoglobin (HbR) map, a total hemoglobin (HbT) map, and an oxygen saturation (StO₂) map for a region of interest (ROI) of the tissue of the subject;
displaying, via a graphical user interface (GUI) stored on a machine-readable medium in operable communication with the NIR optical imager, the dynamic maps;
analyzing the dynamic maps; and
determining, based on the dynamic maps, a likelihood that the subject has vascular calcification (VC) in the heart,
the filter being a long-pass filter or a band-pass filter,
the subject being a human subject,
the vascular occlusion protocol comprising providing an external stimulus configured to alter peripheral tissue oxygenated flow under skin of the subject,
the at least one NIR light of the NIR optical imager being a dual-wavelength light configured to emit light at a first wavelength and a second wavelength different from the first wavelength,
each of the first wavelength and the second wavelength being in a range of from 650 nanometers (nm) to 950 nm,
the at least one NIR light of the NIR optical imager being a light-emitting diode (LED),
the NIR optical imager further comprising an LED driver configured to multiplex light from the at least one NIR light, and
the method further comprising multiplexing the first wavelength and the second wavelength at a first temporal frequency and a second temporal frequency, respectively,
the first temporal frequency being the same as the second temporal frequency,
the first temporal frequency being in a range of from 0.5 Hertz (Hz) to 100 Hz,
the method further comprising:
analyzing the dynamic maps;
generating flow correlation maps from the dynamic maps;
analyzing the spatio-temporal diffuse reflected maps; and
determining the likelihood that the subject has vascular calcification (VC) in the heart based on at least one of the following: an extent of change in a hemoglobin parameter, or its related derivative, from the dynamic maps; the spatio-temporal diffuse reflected maps; a rate of occlusion of a hemoglobin parameter, or its related derivative, measured from the dynamic maps in near-real-time; and the flow correlation maps, and
changes in parameters of the dynamic maps being independent of a color of the skin of the subject.
